(19) United States
(12) Patent Application Publication
(10) Pub. No.: US 2025/0239366 A1
(43) Pub. Date: Jul. 24, 2025

(54) SYSTEM, METHOD, AND COMPUTER PROGRAM PRODUCT FOR MANAGING AUTOMATED HEALTHCARE DATA APPLICATIONS USING ARTIFICIAL INTELLIGENCE

(71) Applicant: Bayer Healthcare LLC, Whippany, NJ (US)

(72) Inventors: Padma Ayodhimani, Dublin, OH (US); Venkat Ramamurthy, Dresher, PA (US); Francesco DESENSI, Mars, PA (US)

(21) Appl. No.: 18/838,918

(22) PCT Filed: Mar. 14, 2023
(86) PCT No.: PCT/US2023/064290

§ 371 Date: Aug. 15, 2024

Related U.S. Application Data
(60) Provisional application No. 63/319,983, filed on Mar. 15, 2022.

(51) Int. Cl. G16H 50/20; G16H 15/00; G16H 50/70

(52) U.S. Cl. CPC G16H 50/20; G16H 15/00; G16H 50/70

(57) ABSTRACT

Provided is a system for managing automated healthcare data applications using artificial intelligence (AI) that includes at least one processor programmed or configured to receive healthcare data from a data source, determine a classification of the healthcare data using a machine learning model, wherein the machine learning model is configured to provide a predicted classification of an automated healthcare data analysis application of a plurality of automated healthcare data analysis applications based on an input, and provide the healthcare data to an automated healthcare data analysis application based on the classification of the healthcare data. Methods and computer program products are also disclosed.

### CROSS-REFERENCE TO RELATED APPLICATIONS

This application claims the benefit of U.S. Provisional Application No. 63/319,983, filed Mar. 15, 2022, the entire disclosure of which is hereby incorporated by reference in its entirety.

### BACKGROUND

### Field

This disclosure relates generally to managing health informatics applications and, in some non-limiting embodiments, to systems, methods, and computer program products that provide for managing automated healthcare data analysis applications using artificial intelligence, in particular, machine learning models.

### Technical Considerations

Health informatics may refer to a field of science and engineering that develops methods and technologies for acquiring, processing, and/or studying of healthcare data (e.g., medical data, patient data, etc.) associated with a patient. In some instances, the healthcare data may come from different sources and/or modalities, such as electronic medical records (e.g., electronic health records), diagnostic test results, and/or medical scans. Health informatics applications may include solutions to problems encountered with medical data and analysis of such medical data using computational techniques.

Artificial intelligence (AI) may be used in healthcare as a way to mimic human cognition in analysis, presentation, and/or comprehension of healthcare data. AI may describe the ability of computer programs, such as computer algorithms, to approximate conclusions based on input data, which may be medical data. In some instances, computer algorithms can be used to recognize patterns in data and create logic for identifying such patterns. Such computer algorithms may include machine learning models that are trained to perform certain tasks using extensive amounts of input data. In some instances, health informatics applications may include AI based health informatics applications.

However, health informatics applications may be implemented in a setting where complex programming and processing resources are required to provide input data, in the form of healthcare data associated with a patient, to the health informatics applications for data analysis tasks. Accordingly, manually programmed processes that are developed for providing the input data to the health informatics applications may require intensive amounts of network resources, may require constant manual updating, and may be inaccurate.

### SUMMARY

Accordingly, disclosed are systems, methods, and computer program products that provide for managing automated healthcare data analysis applications using artificial intelligence (AI), in particular, machine learning models.

Clause 1: A system for managing automated healthcare data analysis applications using artificial intelligence (AI), comprising: at least one processor programmed or configured to: receive healthcare data from a data source; determine a classification of the healthcare data using a machine learning model, wherein the machine learning model is configured to provide a predicted classification of an automated healthcare data analysis application of a plurality of automated healthcare data analysis application based on an input; and provide the healthcare data to an automated healthcare data analysis application based on the classification of the healthcare data.

Clause 2: The system of clause 1, wherein the at least one processor is further programmed or configured to: transmit an output of the automated healthcare data analysis application to a destination system.

Clause 3: The system of clause 1 or 2, wherein the destination system comprises: a picture archiving and communication system (PACS) an electronic medical record (EMR) system; a data reporting system; a communication device associated with a medical device; or a user device associated with a patient.

Clause 4: The system of any of clauses 1-3, wherein the at least one processor is further programmed or configured to: train the machine learning model based on historic healthcare data from a plurality of data sources.

Clause 5: The system of any of clauses 1-4, wherein the machine learning model is configured to receive a data record as an input, wherein the data record comprises at least one feature, wherein the at least one feature comprises: at least one feature associated with anatomical aspects of a body; at least one feature associated with a protocol of a device; at least one feature associated with a characteristic of natural language processing; at least one feature associated with a manual configuration of a device; or any combination thereof.

Clause 6: The system of any of clauses 1-5, wherein the machine learning model is configured to determine whether the at least one feature comprises: a feature of a category associated with anatomical aspects of a body; a feature of a category associated with protocol of a device; a feature of a category associated with a characteristic of natural language processing; a feature of a category associated with a manual configuration of a device; or any combination thereof.

Clause 7: The system of any of clauses 1-6, wherein the data source comprises: an electronic medical record (EMR) system; a medical imaging system; a communication device associated with a medical device; a fluid injection system; a pathology information system; a laboratory information system; or a user device associated with a patient.

Clause 8: The system of any of clauses 1-7, wherein the automated healthcare data analysis application comprises an AI based healthcare data analysis application.

Clause 9: A computer program product for managing automated healthcare data analysis applications using artificial intelligence (AI), the computer program product comprising at least one non-transitory computer-readable medium including one or more instructions that, when executed by at least one processor, cause the at least one processor to: receive healthcare data from a data source; determine a classification of the healthcare data using a machine learning model, wherein the machine learning model is configured to provide a predicted classification of an automated healthcare data analysis application of a plurality of automated healthcare data analysis application based on an input; and provide the healthcare data to an automated healthcare data analysis application based on the classification of the healthcare data.

Clause 10: The computer program product of clause 9, wherein the one or more instructions further cause the at least one processor to: transmit an output of the automated healthcare data analysis application to a destination system.

Clause 11: The computer program product of clause 9 or 10, wherein the destination system comprises: a picture archiving and communication system (PACS) an electronic medical record (EMR) system; a data reporting system; a communication device associated with a medical device; or a user device associated with a patient.

Clause 12: The computer program product of any of clauses 9-11, wherein the one or more instructions further cause the at least one processor to: train the machine learning model based on historic healthcare data from a plurality of data sources.

Clause 13: The computer program product of any of clauses 9-12, wherein the machine learning model is configured to receive a data record as an input, wherein the data record comprises at least one feature, wherein the at least one feature comprises: at least one feature associated with anatomical aspects of a body; at least one feature associated with a protocol of a device; at least one feature associated with a characteristic of natural language processing; at least one feature associated with a manual configuration of a device; or any combination thereof.

Clause 14: The computer program product of any of clauses 9-13, wherein the machine learning model is configured to determine whether the at least one feature comprises: a feature of a category associated with anatomical aspects of a body; a feature of a category associated with protocol of a device; a feature of a category associated with a characteristic of natural language processing; a feature of a category associated with a manual configuration of a device; or any combination thereof.

Clause 15: The computer program product of any of clauses 9-14, wherein the data source comprises: an electronic medical record (EMR) system; a medical imaging system; a communication device associated with a medical device; a fluid injection system; a pathology information system; a laboratory information system; or a user device associated with a patient.

Clause 16: The computer program product of any of clauses 9-15, wherein the automated healthcare data analysis application comprises an artificial intelligence (AI) based healthcare data analysis application.

Clause 17: A method for managing automated healthcare data analysis applications using artificial intelligence, comprising: receiving healthcare data from a data source; determining a classification of the healthcare data using a machine learning model, wherein the machine learning model is configured to provide a predicted classification of an automated healthcare data analysis application of a plurality of automated healthcare data analysis application based on an input; and providing the healthcare data to an automated healthcare data analysis application based on the classification of the healthcare data.

Clause 18: The method of clause 17, further comprising: transmit an output of the automated healthcare data analysis application to a destination system.

Clause 19: The method of clause 17 or 18, further comprising: training the machine learning model based on historic healthcare data from a plurality of data sources.

Clause 20: The method of any of clauses 17-19, wherein the machine learning model is configured to receive a data record as an input, wherein the data record comprises at least one feature, wherein the at least one feature comprises: at least one feature associated with anatomical aspects of a body; at least one feature associated with a protocol of a device; at least one feature associated with a characteristic of natural language processing; at least one feature associated with a manual configuration of a device; or any combination thereof.

These and other features and characteristics of the present disclosure, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the present disclosure. As used in the specification and the claims, the singular form of “a,” “an,” and “the” include plural referents unless the context clearly dictates otherwise.

### BRIEF DESCRIPTION OF THE DRAWINGS

Additional advantages and details of non-limiting embodiments are explained in greater detail below with reference to the exemplary embodiments that are illustrated in the accompanying schematic figures, in which:

FIG. 1 is a diagram of a non-limiting embodiment of an environment in which systems, methods, and/or computer program products described herein, may be implemented according to the present disclosure;

FIG. 2 is a diagram of a non-limiting embodiment of components of one or more devices and/or one or more systems of FIG. 1;

FIG. 3 is a flowchart of a non-limiting embodiment of a process for managing automated healthcare data analysis applications using artificial intelligence (AI);

FIG. 4 is a diagram of a non-limiting embodiment of an AI data management system; and

FIGS. 5A-5C are diagrams of a non-limiting embodiment of an implementation of a process for managing automated healthcare data analysis applications using AI.

### DESCRIPTION

For purposes of the description hereinafter, the terms “end,” “upper,” “lower,” “right,” “left,” “vertical,” “horizontal,” “top,” “bottom,” “lateral,” “longitudinal,” and derivatives thereof shall relate to the disclosure as it is oriented in the drawing figures. However, it is to be understood that the disclosure may assume various alternative variations and step sequences, except where expressly specified to the contrary. It is also to be understood that the specific devices and processes illustrated in the attached drawings, and described in the following specification, are simply exemplary embodiments of the disclosure. Hence, specific dimensions and other physical characteristics related to the embodiments of the embodiments, disclosed herein, are not to be considered as limiting unless otherwise indicated.

No aspect, component, element, structure, act, step, function, instruction, and/or the like used herein should be construed as critical or essential unless explicitly described as such. Also, as used herein, the articles “a” and “an” are intended to include one or more items, and may be used interchangeably with “one or more” and “at least one.” Furthermore, as used herein, the term “set” is intended to include one or more items (e.g., related items, unrelated items, a combination of related and unrelated items, and/or the like) and may be used interchangeably with “one or more” or “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise. The phrase “based on” may also mean “in response to” where appropriate.

As used herein, the terms “communication” and “communicate” may refer to the reception, receipt, transmission, transfer, provision, and/or the like of information (e.g., data, signals, messages, instructions, commands, and/or the like). For one unit (e.g., a device, a system, a component of a device or system, combinations thereof, and/or the like) to be in communication with another unit means that the one unit is able to directly or indirectly receive information from and/or transmit information to the other unit. This may refer to a direct or indirect connection that is wired and/or wireless in nature. Additionally, two units may be in communication with each other even though the information transmitted may be modified, processed, relayed, and/or routed between the first and second unit. For example, a first unit may be in communication with a second unit even though the first unit passively receives information and does not actively send information to the second unit. As another example, a first unit may be in communication with a second unit if at least one intermediary unit (e.g., a third unit located between the first unit and the second unit) processes information received from the first unit and sends the processed information to the second unit. In some non-limiting embodiments, information may refer to a network packet (e.g., a data packet and/or the like) that includes data.

Systems, methods, and computer program products are disclosed that provide solutions to the above mentioned challenges. For example, as disclosed herein, a system for managing automated healthcare data analysis applications using artificial intelligence (AI) may include at least one processor programmed or configured to receive healthcare data from a data source, determine a classification of the healthcare data using a machine learning model, wherein the machine learning model is configured to a provide a predicted classification of an automated healthcare data analysis application of a plurality of automated healthcare data analysis application based on an input, and provide the healthcare data to an automated healthcare data analysis application based on the classification of the healthcare data.

In some non-limiting embodiments, the at least one processor is further programmed or configured to transmit an output of the automated healthcare data analysis application to a destination system. In some non-limiting embodiments, the destination system comprises a picture archiving and communication system (PACS), an electronic medical record (EMR) system, a data reporting system, a communication device associated with a medical device, and/or a user device associated with a patient. In some non-limiting embodiments, the at least one processor is further programmed or configured to train the machine learning model based on healthcare data from a plurality of data sources.

In some non-limiting embodiments, the machine learning model is configured to receive an data record as an input, wherein the data record comprises at least one feature, wherein the at least one feature comprises: at least one feature associated with anatomical aspects of a body, at least one feature associated with a protocol of a device, at least one feature associated with a characteristic of natural language processing, and/or at least one feature associated with a manual configuration of a device. In some non-limiting embodiments, the machine learning model is configured to determine whether the at least one feature comprises a feature of a category associated with anatomical aspects of a body, a feature of a category associated with protocol of a device, a feature of a category associated with a characteristic of natural language processing, and/or a feature of a category associated with a manual configuration of a device.

In some non-limiting embodiments, the data source comprises an EMR system, a medical imaging system, a communication device associated with a medical device, a fluid injection system, a pathology information system, a laboratory information system, and/or a user device associated with a patient. In some non-limiting embodiments, an automated healthcare data analysis application comprises an AI based healthcare data analysis application.

In this way, the present disclosure provides a system for managing automated healthcare data analysis applications using AI that reduces an amount of network resource required as compared to a manually programmed process. Additionally, the system reduces a need for manual updating process rules for managing automated healthcare data analysis applications and improves accuracy with regard to how healthcare data is provided to automated healthcare data analysis applications.

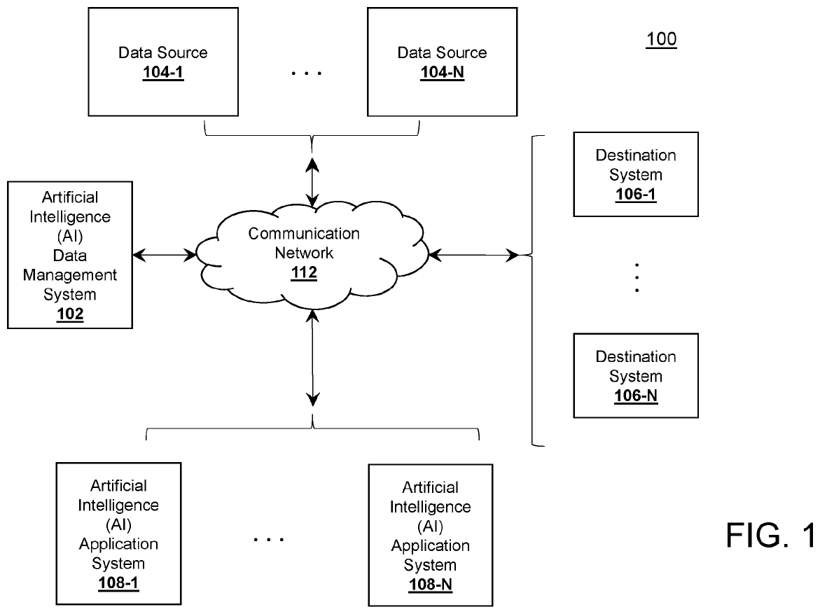

Referring now to FIG. 1, FIG. 1 is a diagram of a non-limiting embodiment of an environment 100 in which devices, systems, methods, and/or computer program products, described herein, may be implemented. As shown in FIG. 1, environment 100 includes artificial intelligence (AI) data management system 102, data sources 104-1 through 104-N (referred to hereafter individually as data source 104, or together as data sources 104, where appropriate), destination systems 106-1 through 106-N (referred to hereafter individually as destination system 106, or together as destination systems 106, where appropriate), and AI application systems 108-1 through 108-N (referred to hereafter individually as AI application system 108, or together as AI application systems 108, where appropriate).

AI data management system 102 may interconnect (e.g., establish a connection to communicate with and/or the like) with data sources 104, destination systems 106, and/or AI application systems 108 via communication network 112. In some non-limiting embodiments, AI data management system 102 may interconnect (e.g., establish a connection to communicate with and/or the like) with data sources 104, destination systems 106, and/or AI application systems 108 via wired connections, wireless connections, or a combination of wired and wireless connections.

In some non-limiting embodiments, AI data management system 102 may include one or more devices capable of being in communication with data sources 104, destination systems 106, and/or AI application system 108 via communication network 112. For example, AI data management system 102 may include a server, a group of servers, a telecommunications gateway (e.g., a network gateway), a universal gateway, a router, and/or other like devices. Additionally or alternatively, AI data management system 102 may include a computing device, such as a desktop computer, a mobile device (e.g., a tablet, a smartphone, a wearable device, etc.), and/or the like. In some non-limiting embodiments, AI data management system 102 may include one or more (e.g., a plurality of) applications (e.g., software applications) that perform a set of functionalities on an external application programming interface (API) to send data to an external system, such as destination system 106 or AI application system 108, associated with the external API and to receive data from the external system associated with the external API. In some non-limiting embodiments, AI data management system 102 may include one or more subsystems. For example, AI data management system 102 may include one or more subsystems that performs anatomical intelligence (e.g., body part detection), one or more subsystems that performs modality detection (e.g., a modality of a patient procedure associated with healthcare data), and/or one or more subsystems that performs text analysis intelligence (e.g., identification and analysis of words or phrases, such as natural language processing). By way of example, AI data management system 102 may include a smart AI router that accepts imaging data and other related health care data from multiple sources that are relevant to the patient's imaging data. The data may include DICOM data, Health Level Seven (HL7) Order information, EMR data, Lab data, Prescription (e.g., Rx) information, Prior AI reports and additional reports from AI, Pathology data, wearable data, health care billing codes, and other healthcare data.

DICOM data may include inputs used for processing. DICOM data can be sent to the smart AI router from a PACS or modality (e.g., device included in a healthcare system, such as a fluid injection system) or it can be retrieved from the modality and/or PACS. The DICOM data could be from any medical equipment (e.g., medical imaging equipment), not limited to CT, MRI, X-Ray, Mammograms, Ultrasound, Positron Emission Tomography (PET) Exams, and/or Single Photon Emission Computed Tomography exams. HL7 Order information may include data that can provide additional detail about an imaging study, such as a reason for an exam. EMR data may include data that can provide additional detail about the patient's health history such as tobacco use (e.g., smoking history). Lab data may include data that provides information that can be used in additional to image data to assess relevant disease conditions. Prescription information may include data that identifies the medication a patient is taking/taken that can be used to assess the relevancy for an AI process to track disease progression e.g., for clinical trial eligibility/efficacy of the medication as assessed by imaging data. Prior AI reports and additional reports from AI may include data regarding one AI application's output to determine what other AI application to which to send a study. For example, a lung nodule detection AI application may provide an output that is sent to a nodule characterization AI application. Wearable data may include data from a continuous glucose monitoring device, a single lead EKG device, and/or the like. Other health data may include data from other data sources that track a patient's health info that could be relevant for disease management use cases.

In such an example, the smart AI router may include an image analyzer module that detects the body parts and its anatomical context (e.g., body part, such as a lung, and anatomical context, such as carina, prone or supine positioning, etc.) present in the imaging data using pixel information, and in some instances, without relying on the DICOM header data like study descriptions. The image analyzer module may work the same with both anonymized and non-anonymized data as this is based on the pixel data.

The image analyzer module may check to see if the imaging data has all the series and/or images that are required to consider a study complete as per a protocol used to acquire the images. For example, if the study is a brain MRI, the image analyzer modules checks to see if T1, T2 diffusion weighted imaging (DWI) series are present for a brain tumor evaluation protocol. Additionally, the smart AI router analyzes the images to determine if contrast has been applied or not to distinguish AI applications that require contrast-enhanced exams for successful processing. The image analyzer module also checks for the image quality to ensure that the imaging data will be useful if sent to the AI for processing.

The smart AI router may include a matching module that takes an output of the image analyzer module and compares the output to the input requirement parameters of all the AI applications for a body region to identify an AI application that can be eligible for processing. The matching module has a database of AI applications and corresponding input requirements. The matching module provides an administrator with a user interface to view and add new AI application or change parameters as needed.

The matching module may compare ae list of eligible AI applications to be processed against the customer's guidelines for processing. Customer guidelines could be driven by need to restrict unnecessary processing or processing that cannot be reimbursed and/or paid for by the patient. The smart AI router saves the applied customers guidelines as presets to use for subsequent processing. In the absence of customer specific guidelines on AI application usage, the matching module will match the list against standard guidelines (e.g., country/regional/industry standard guidelines), such as lung cancer screening guidelines by the US Center of Medicare/Medicaid service (CMS) that recommend patients age over 50 and tobacco smoking history of at least 20 packs per year to get a screening. The matching module has a database of guidelines (e.g., customer specific and/or industry specific standard) and provides an administrator with a user interface to view and add new AI application or change guidelines as needed. In the example of CMS guidelines, the CMS guidelines were changed from 55 years to 50 and the user interface allows the guidelines to be updated when they change. A Guideline's input can also come from other AI application processing.

The smart AI router may include a recommendation module that assesses disease states that need to be evaluated by an AI application for diagnosis, detection, quantification or for opportunistic screening based on a patient's health data or for outcomes measurement. In some examples, the recommendation module may operate in the absence of a customer or industry guideline.

The smart AI router may assign priority for AI processing. An AI application that aids in diagnosis is run first and any opportunistic screening is scheduled later by the smart AI router to allow for optimal processing bandwidth utilization. The smart AI router allows for easy configuration of the recommendation module for existing disease uses cases and adding new disease management use cases. The smart AI router provides a framework where health data points from new data sources or additional data points from existing sources can be added/changed in the module's logic. The smart AI router provides a way to monitor the performance of the solution and to learn from the usage.

In some examples, the smart AI router may include a continuous learning module where the smart AI router learns from the results of past recommendations. For example, a radiology report, patient's diagnosis, and/or disease progression is tracked for every recommendation decision as an input to continuous learning. In such an example, if the recommendation module did not recommend lung cancer screening but subsequent Chest CT's found cancerous lung nodules or vice versa, the smart AI router may update as appropriate. In some examples, the smart AI router may include a smart protocol module to ensure input parameter requirements are met for successful AI application processing for a disease management use case. An input to this process is patient imaging scheduling data, such as radiation and contrast dose management and patient healthcare, including data from contrast injectors. The smart protocol module will evaluate the AI needed based on the reason for exam and identify additional screening opportunities that can be of value to the patient and recommend protocols to meet these screenings.

A disease management use case framework may include receiving a type of imaging data type and detecting a body part. Disease conditions may be mapped to the image data type and body part and for each disease condition an AI application that can aid in the detection and/or quantification of the disease may be identified, along with input parameters of the AI application, industry guideline, a customer guideline, and/or a recommendation rule (e.g., a Calantic recommendation rule), healthcare data used in determining relevancy for opportunistic screening and the source of the data. In this way, the smart AI router may use a reason for exam to drives the AI application usage. In addition, the smart AI router (e.g., a recommendation module) will suggest additional relevant AI based on health data or in the absence of reason for exam and priority for the AI processing.

An example of a Thoracic disease management use case framework may include receiving imaging data of a type that includes contrast and non-contrast Chest CT/Both Lungs detected in full. The Disease conditions mapped to the image data type include lung cancer, cardiac calcification, pulmonary embolism (PE), chronic obstructive pulmonary disease (COPD)/interstitial lung disease (ILD), and/or chronic thromboembolic pulmonary hypertension (CTEPH).

For each disease condition, such as lung cancer, an AI application that can aid in the detection and/or quantification of the disease may operate by analyzing a clear read CT. The input parameters of the AI application include a non-contrast Chest CT at greater than 5 mm slice thickness. An industry guideline for the analysis may include to automatically screen a patient at an age greater than or equal to 50 that smokes 20 packs of tobacco per year. A customer guideline may be absent and a recommendation may receive, as input, a reason for an exam being a suspected pulmonary embolism, high blood pressure from EMR data, smoking history and age greater than or equal to 50, and/or an EMR A1C value indicating pre-diabetes. The smart AI router may determine to run a PE AI application at a high priority, determine to run a CTEPH AI application as a lower priority, determine to run a clear read for lung nodule detection AI application as a medium priority, and recommend a cardiac artery calcification (CAC) score evaluation as a low priority.

In some non-limiting embodiments, data source 104 may include one or more devices capable of being in communication with AI data management system 102, destination system 106, and AI application system 108 via communication network 112 and be capable of performing fluid injection procedures. For example, data source 104 may include a server, a computing device, such as a desktop computer, a mobile device (e.g., a tablet, a smartphone, a wearable, such as a wearable health sensor, etc.), and/or the like. In some non-limiting embodiments, data source 104 may include a hospital information system (HIS), an EMR system, a medical imaging system (e.g., an imaging scanner), a fluid injection system (e.g., a fluid injector), a device associated with a facility, such as a communication device associated with a medical device (e.g., a hand-held medical device, a wearable medical device, such as a portable health sensor, etc.), a fluid injection system, a pathology information system, a laboratory information system, and/or a device associated with a patient (e.g., a user device, such as a computing device operated by a patient).

In some non-limiting embodiments, a fluid injection system may include one or more injection devices (e.g., one or more fluid injection devices). In some non-limiting embodiments, a fluid injection system is configured to administer (e.g., inject, deliver, etc.) contrast fluid including a contrast agent to a patient, and/or administer an aqueous fluid, such as saline, to a patient before, during, and/or after administering the contrast fluid. For example, a fluid injection system can inject one or more prescribed dosages of contrast fluid directly into a patient's blood stream via a hypodermic needle and syringe. In some non-limiting embodiments, a fluid injection system may be configured to continually administer the aqueous fluid to a patient through a peripheral intravenous line (PIV) and catheter, and one or more prescribed dosages of contrast fluid may be introduced into the PIV and administered via the catheter to the patient. In some non-limiting embodiments, a fluid injection system is configured to inject a dose of contrast fluid followed by administration of a particular volume of the aqueous fluid. In some non-limiting embodiments, a fluid injection system may include one or more exemplary injection systems or injectors that are disclosed in: U.S. patent application Ser. No. 09/715,330, filed on Nov. 17, 2000, issued as U.S. Pat. No. 6,643,537; U.S. patent application Ser. No. 09/982,518, filed on Oct. 18, 2001, issued as U.S. Pat. No. 7,094,216; U.S. patent application Ser. No. 10/825,866, filed on Apr. 16, 2004, issued as U.S. Pat. No. 7,556,619; U.S. patent application Ser. No. 12/437,011, filed May 7, 2009, issued as U.S. Pat. No. 8,337,456; U.S. patent application Ser. No. 12/476,513, filed Jun. 2, 2009, issued as U.S. Pat. No. 8,147,464; and U.S. patent application Ser. No. 11/004,670, filed on Dec. 3, 2004, issued as U.S. Pat. No. 8,540,698, the disclosures of each of which are incorporated herein by reference in their entireties. In some non-limiting embodiments, a fluid injection system may include the MEDRAD® Stellant CT Injection System, the MEDRAD® Stellant Flex CT Injection System, the MEDRAD® MRXperion MR Injection System, the MEDRAD® Mark 7 Arterion Injection System, the MEDRAD® Intego PET Infusion System, or the MEDRAD® Centargo CT Injection System, all of which are provided by Bayer Healthcare LLC.

In some non-limiting embodiments, an HIS may include one or more subsystems, such as a patient procedure tracking system (e.g., a system that operates a modality worklist, a system that provides patient demographic information for fluid injection procedures and/or medical imaging procedures, etc.), a fluid injector management system, an image archive and communication system (e.g., a picture archive and communication system (PACS)), a radiology information system, and/or a radiology analytics system (e.g., the Radimetrics Enterprise Application marketed and sold by Bayer Healthcare LLC).

In some non-limiting embodiments, a medical imaging system may include an ultrasound system, a echocardiography system, a magnetic resonance imaging (MRI) system, an electromagnetic radiation system, (e.g., a conventional 2-D X-ray, a 3-D computed tomography (CT) scanning system, a fluoroscopy system, etc.), capable of communicating via communication network 112 and capable of performing medical imaging procedures, including medical imaging procedures involving the use of a radiological contrast material. In some non-limiting embodiments, a medical imaging system may provide an image of a patient and/or data associated with the image of the patient (e.g., an image of a patient as the result of an imaging study). The data associated with the image of the patient may include data in a digital imaging and communications in medicine (DICOM) format, which may include metadata, pixel data, and/or additional data associated with an imaging procedure that was performed on the patient to provide the image.

In some non-limiting embodiments, a fluid injection system service and control system may include a system designed to provide functionality for a user (e.g., a service representative of a company that sells and/or operates a fluid injection system) to interact with a fluid injection system. For example, fluid injection system service and control system may provide the functionality for the user to perform upgrades, perform restoration operations, logging operations, data backup operations, and/or the like.

In some non-limiting embodiments, a laboratory information system (e.g., a laboratory information management system (LIMS) or laboratory management system (LMS)) may include a system designed to support operations of a laboratory (e.g., a medical laboratory). The laboratory information system may include functionality, such as workflow, data tracking, flexible architecture, and/or data exchange interfaces, which supports the use of a laboratory in regulated environments. In some non-limiting embodiments, the laboratory information system may include an enterprise resource planning tool that is designed to manage aspects of laboratory informatics (e.g., patient data associated with laboratory processes and/or laboratory testing).

In some non-limiting embodiments, a pathology information system may include a system designed to register specimens, record gross and/or microscopic findings, regulate laboratory workflow, formulate and/or sign out a report, disseminate a report to an intended recipient across a health system, support quality assurance measures, and/or the like.

In some non-limiting embodiments, destination system 106 may include one or more devices capable of being in communication with AI data management system 102, data sources 104, and/or AI application system 108 via communication network 112. For example, destination system 106 may include a server, a group of servers, a computing device, such as a desktop computer, a mobile device (e.g., a tablet, a smartphone, a wearable device, etc.), and/or the like. In some non-limiting embodiments, destination system 106 may include one or more (e.g., a plurality of) applications (e.g., software applications) that perform a set of functionalities, including workflow activation and results delivery. In some non-limiting embodiments, destination system 106 may include one or more applications that perform the set of functionalities on an external API to send data to an external system, such as AI data management system 102 or AI application system 108, associated with the external API and to receive data from the external system associated with the external API. In some non-limiting embodiments, destination system 106 may include a picture archiving and PACS, an EMR system, a reporting system (e.g., a system that generates medical reports based on healthcare data), a device associated with a facility (e.g., a hospital), and/or a device associated with a patient.

In some non-limiting embodiments, AI application system 108 may include one or more devices capable of being in communication with AI data management system 102, data sources 104, and/or destination system 106 via communication network 112. For example, AI application system 108 may include a server, a group of servers, a computing device, such as a desktop computer, a mobile device (e.g., a tablet, a smartphone, a wearable device, etc.), and/or the like. In some non-limiting embodiments, AI application system 108 may include one or more automated healthcare data analysis applications. For example, AI application system 108 may include one or more automated healthcare data analysis applications stored in a memory device of AI application system 108. In another example, AI application system 108 may include one or more automated healthcare data analysis applications that are each stored on a device (e.g., a server). In some non-limiting embodiments, the one or more automated healthcare data analysis applications may perform a set of functionalities on an external API to send data to an external system, such as AI data management system 102 or another AI application system 108, associated with the external API, and/or to receive data from the AI application system 108 associated with the external API.

In some non-limiting embodiments, an automated healthcare data analysis application may include an AI software application that performs an analysis (e.g., automated analysis) of healthcare data. In some non-limiting embodiments, an AI software application may perform an analysis that includes a prediction of a diagnosis regarding a medical condition. Additionally or alternatively, an AI software application may perform an analysis that includes a recommendation regarding a treatment for a patient.

In some non-limiting embodiments, communication network 112 may include one or more wired and/or wireless networks. For example, communication network 112 may include a cellular network (e.g., a long-term evolution (LTE) network, a third generation (3G) network, a fourth generation (4G) network, a fifth generation (5G) network, a code division multiple access (CDMA) network, and/or the like), a local area network (LAN), a wide area network (WAN), a wireless LAN (WLAN), a private network, an ad hoc network, an intranet, the Internet, a fiber optic-based network, an Ethernet network, a universal serial bus (USB) network, a cloud computing network, and/or the like, and/or a combination of some or all of these or other types of networks.

The number and arrangement of systems and/or devices shown in FIG. 1 are provided as an example. There may be additional systems and/or devices, fewer systems and/or devices, different systems and/or devices, or differently arranged systems and/or devices than those shown in FIG. 1. Furthermore, two or more systems and/or devices shown in FIG. 1 may be implemented within a single system or a single device, or a single system or a single device shown in FIG. 1 may be implemented as multiple, distributed systems or devices. Additionally or alternatively, a set of systems or a set of devices (e.g., one or more systems, one or more devices) of environment 100 may perform one or more functions described as being performed by another set of systems or another set of devices of environment 100.

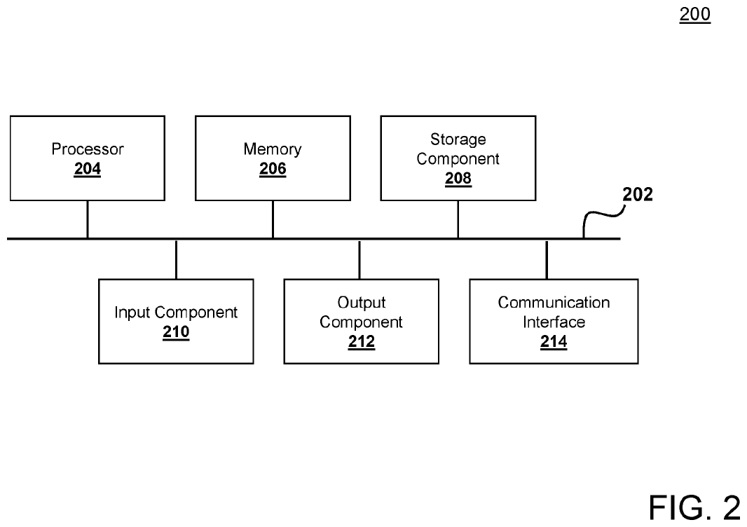

Referring now to FIG. 2, FIG. 2 is a diagram of example components of device 200. Device 200 may correspond to AI data management system 102, data source 104, destination system 106, and/or AI application system 108. In some non-limiting aspects or embodiments, AI data management system 102, data source 104, destination system 106, and/or AI application system 108 may include at least one device 200 and/or at least one component of device 200. As shown in FIG. 2, device 200 may include bus 202, processor 204, memory 206, storage component 208, input component 210, output component 212, and communication component 214.

Bus 202 may include a component that permits communication among the components of device 200. In some non-limiting embodiments, processor 204 may be implemented in hardware, software, or a combination of hardware and software. For example, processor 204 may include a processor (e.g., a central processing unit (CPU), a graphics processing unit (GPU), an accelerated processing unit (APU), and/or the like), a microprocessor, a digital signal processor (DSP), and/or any processing component (e.g., a field-programmable gate array (FPGA), an application-specific integrated circuit (ASIC), and/or the like) that can be programmed to perform a function. Memory 206 may include random access memory (RAM), read-only memory (ROM), and/or another type of dynamic or static storage device (e.g., flash memory, magnetic memory, optical memory, and/or the like) that stores information and/or instructions for use by processor 204.

Storage component 208 may store information and/or software related to the operation and use of device 200. For example, storage component 208 may include a hard disk (e.g., a magnetic disk, an optical disk, a magneto-optic disk, a solid state disk, and/or the like), a compact disc (CD), a digital versatile disc (DVD), a floppy disk, a cartridge, a magnetic tape, and/or another type of computer-readable medium, along with a corresponding drive.

Input component 210 may include a component that permits device 200 to receive information, such as via user input (e.g., a touchscreen display, a keyboard, a keypad, a mouse, a button, a switch, a microphone, a camera, and/or the like). Additionally or alternatively, input component 210 may include a sensor for sensing information (e.g., a global positioning system (GPS) component, an accelerometer, a gyroscope, an actuator, and/or the like). Output component 212 may include a component that provides output information from device 200 (e.g., a display, a speaker, one or more light-emitting diodes (LEDs), and/or the like).

Communication component 214 may include a transceiver-like component (e.g., a transceiver, a separate receiver and transmitter, and/or the like) that enables device 200 to communicate with other devices, such as via a wired connection, a wireless connection, or a combination of wired and wireless connections. Communication component 214 may permit device 200 to receive information from another device and/or provide information to another device. For example, communication component 214 may include an Ethernet interface, an optical interface, a coaxial interface, an infrared interface, a radio frequency (RF) interface, a universal serial bus (USB) interface, a Wi-Fi® interface, a cellular network interface, and/or the like.

Device 200 may perform one or more processes described herein. Device 200 may perform these processes based on processor 204 executing software instructions stored by a computer-readable medium, such as memory 206 and/or storage component 208. A computer-readable medium (e.g., a non-transitory computer-readable medium) is defined herein as a non-transitory memory device. A non-transitory memory device includes memory space located inside of a single physical storage device or memory space spread across multiple physical storage devices.

Software instructions may be read into memory 206 and/or storage component 208 from another computer-readable medium or from another device via communication component 214. When executed, software instructions stored in memory 206 and/or storage component 208 may cause processor 204 to perform one or more processes described herein. Additionally or alternatively, hardwired circuitry may be used in place of or in combination with software instructions to perform one or more processes described herein. Thus, embodiments described herein are not limited to any specific combination of hardware circuitry and software.

Memory 206 and/or storage component 208 may include data storage or one or more data structures (e.g., a database and/or the like). Device 200 may be capable of retrieving information from, storing information in, or searching information stored in the data storage or one or more data structures in memory 206 and/or storage component 208.

The number and arrangement of components shown in FIG. 2 are provided as an example. In some non-limiting embodiments, device 200 may include additional components, fewer components, different components, or differently arranged components than those shown in FIG. 2. Additionally or alternatively, a set of components (e.g., one or more components) of device 200 may perform one or more functions described herein as being performed by another set of components of device 200.

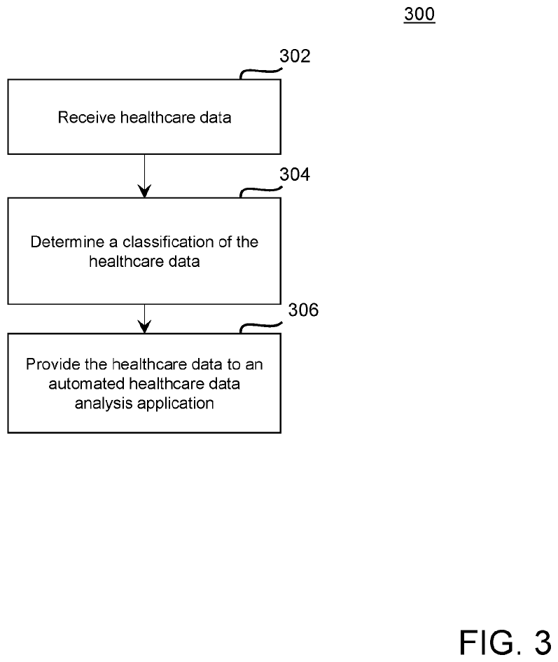

Referring now to FIG. 3, FIG. 3 is a flowchart of a non-limiting embodiment of a process 300 for managing automated healthcare data analysis applications using AI. In some non-limiting embodiments, one or more of the parts of process 300 may be performed (e.g., completely, partially, etc.) by AI data management system 102 (e.g., one or more devices of AI data management system 102). In some non-limiting embodiments, one or more of the parts of process 300 may be performed (e.g., completely, partially, etc.) by another device or a group of devices separate from or including AI data management system 102, data source 104, destination system 106, and/or AI application system 108.

As shown in FIG. 3, at part 302, process 300 includes receiving healthcare data from a data source. For example, AI data management system 102 may receive healthcare data from one or more data sources 104. In some non-limiting embodiments, AI data management system 102 may receive the healthcare data based on a device associated with data source 104 performing a procedure. For example, AI data management system 102 may receive the healthcare data after the device associated with data source 104 performs a procedure. In some non-limiting embodiments, AI data management system 102 may receive the healthcare data from a device associated with a hospital (e.g., a server operated by a hospital, such as a server that is on-site at a hospital or a server that is located remotely from a hospital, a hospital information system, etc.). In some non-limiting embodiments, AI data management system 102 may store the healthcare data (e.g., with an application, in a memory device, etc.) based on receiving the healthcare data.

In some non-limiting embodiments, AI data management system 102 may receive the healthcare data and generate healthcare data associated with classifications of automated healthcare data analysis applications. For example, AI data management system 102 may receive the healthcare data and determine specific parameters of the healthcare data that are associated with each automated healthcare data analysis application of a plurality of automated healthcare data analysis applications. AI data management system 102 may generate the healthcare data associated with classifications of automated healthcare data analysis applications based on the specific parameters of the healthcare data.

In some non-limiting embodiments, AI data management system 102 may receive the healthcare data (e.g., from a hospital information system) according to a communications protocol for communicating the data via a communication network. For example, AI data management system 102 may receive the healthcare data according to a DICOM communications protocol, according to a HL7 standard communications protocol, and/or the like.

In some non-limiting embodiments, the healthcare data may include data associated with an electronic record (e.g., an electronic patient record) from an EMR system, data associated with an image generated by a medical imaging system, data associated a biometric measurement of a patient, data associated with a fluid injection procedure performed by a fluid injection system, data associated with a record from a pathology information system, and/or a laboratory information system. Additionally or alternatively, healthcare data may include data associated with identification of a patient, data associated with a patient procedure (e.g., data regarding a procedure performed on a patient, such as a patient examination procedure, a fluid injection procedure, a medical imaging procedure, radiation dosage etc.), data associated with a device that is involved in a patient procedure (e.g., data associated with a fluid injection system, data associated with a device of a patient, etc.), and/or the like. In some non-limiting embodiments, data associated with a patient procedure may include data associated with a fluid injection procedure, which may include data associated with a contrast fluid provided during a fluid injection procedure, a gauge of a catheter used during a fluid injection procedure, a fluid injection protocol for a fluid injection procedure, a configuration of a fluid injection system, and/or the like. Additionally or alternatively, data associated with a patient procedure may include data associated with a medical imaging procedure, which may include data associated with an image (e.g., a radiology image), data associated with radiation dosage, and/or the like.

In some non-limiting embodiments, AI data management system 102 may generate a machine learning model (e.g., an automated healthcare data analysis application prediction machine learning model) to provide a prediction (e.g., a prediction that includes a score, such as a risk score) of a classification of an automated healthcare data analysis application of a plurality of automated healthcare data analysis applications based on an input.

In some non-limiting embodiments, the machine learning model may include a machine learning model designed to receive, as an input, healthcare data, and provide, as an output, a predicted classification of an automated healthcare data analysis application. For example, the machine learning model may be designed to receive healthcare data (e.g., data associated with a patient procedure) and provide an output that includes the predicted classification of an automated healthcare data analysis application of a plurality of automated healthcare data analysis applications. The predicted classification of an automated healthcare data analysis application may include a classification of an automated healthcare data analysis application that may be able to provide further analysis of the healthcare data. In some non-limiting embodiments, AI data management system 102 may store the machine learning model (e.g., for later use).

In some non-limiting embodiments, as described herein, AI data management system 102 may process healthcare data (e.g., historical healthcare data associated with automated healthcare data analysis applications, such as healthcare data that includes labels of a specified automated healthcare data analysis application for specific parameters of patient procedures) to obtain training data (e.g., a training dataset) for the machine learning model. For example, AI data management system 102 may process the data to change the data into a format that may be analyzed (e.g., by AI data management system 102) to generate the machine learning model. The data that is changed (e.g., the data that results from the change) may be referred to as training data. In some non-limiting embodiments, AI data management system 102 may process the healthcare data to obtain the training data based on receiving the healthcare data. Additionally or alternatively, AI data management system 102 may process the healthcare data to obtain the training data based on AI data management system 102 receiving an indication, from a user (e.g., a user associated with a user device) of AI data management system 102, that AI data management system 102 is to process the data, such as when AI data management system 102 receives an indication to generate a machine learning model (e.g., for a time interval corresponding to the healthcare data, for a location corresponding to the healthcare data, etc.).

In some non-limiting embodiments, AI data management system 102 may process healthcare data by determining a healthcare data analysis application prediction variable based on the healthcare data. A healthcare data analysis application prediction variable may include a metric, associated with a healthcare data analysis application, which may be derived based on the healthcare data. The healthcare data analysis application prediction variable may be analyzed to generate a machine learning model. For example, the healthcare data analysis application prediction variable may include a variable associated with a patient procedure (e.g., a time of a patient procedure, a protocol used during a patient procedure, an amount of a substance used during a patient procedure, a result of a patient procedure, etc.), a variable associated with an aspect of image (e.g., a radiological image), a variable associated with a patient demographic, a variable associated with a condition of a patient, and/or the like.

In some non-limiting embodiments, AI data management system 102 may analyze the training data to generate the machine learning model. For example, AI data management system 102 may use machine learning techniques to analyze the training data to generate the machine learning model. In some non-limiting embodiments, generating the machine learning model (e.g., based on training data obtained from historical healthcare data associated with automated healthcare data analysis applications) may be referred to as training the machine learning model. The machine learning techniques may include, for example, supervised and/or unsupervised techniques, such as decision trees, random forests, logistic regressions, linear regression, gradient boosting, support-vector machines, extra-trees (e.g., an extension of random forests), Bayesian statistics, learning automata, Hidden Markov Modeling, linear classifiers, quadratic classifiers, association rule learning, and/or the like. In some non-limiting embodiments, the machine learning model may include a model that is specific to a particular characteristic, for example, a model that is specific to a particular location (e.g., a specific location of a hospital), a particular patient demographic, a particular system used during a patient procedure, and/or the like. Additionally or alternatively, the machine learning model may be specific to a particular entity (e.g., a healthcare provider, such as a hospital, a clinical facility, a group of doctors, etc.) that provides healthcare services. In some non-limiting embodiments, AI data management system 102 may generate one or more machine learning models for one or more entities, a particular group of entities, and/or one or more users of one or more entities.

Additionally or alternatively, when analyzing the training data, AI data management system 102 may identify one or more variables (e.g., one or more independent variables) as predictor variables (e.g., features) that may be used to make a prediction when analyzing the training data. In some non-limiting embodiments, values of the predictor variables may be inputs to the machine learning model. For example, AI data management system 102 may identify a subset (e.g., a proper subset) of the variables as the predictor variables that may be used to accurately predict a classification of an automated healthcare data analysis application (e.g., from among a plurality of automated healthcare data analysis applications) to analyze healthcare data. In some non-limiting embodiments, the predictor variables may include one or more of the healthcare data analysis application variables, as discussed above, that have a significant impact (e.g., an impact satisfying a threshold) on a predicted classification of an automated healthcare data analysis application to analyze healthcare data as determined by AI data management system 102.

In some non-limiting embodiments, AI data management system 102 may validate the machine learning model. For example, AI data management system 102 may validate the machine learning model after AI data management system 102 generates the machine learning model. In some non-limiting embodiments, AI data management system 102 may validate the machine learning model based on a portion of the training data to be used for validation. For example, AI data management system 102 may partition the training data into a first portion and a second portion, where the first portion may be used to generate the machine learning model, as described above. In this example, the second portion of the training data (e.g., the validation data) may be used to validate the machine learning model.

In some non-limiting embodiments, AI data management system 102 may validate the machine learning model by providing validation data associated with a user (e.g., healthcare data associated with automated healthcare data analysis applications) as input to the machine learning model, and determining, based on an output of the machine learning model, whether the machine learning model correctly, or incorrectly, predicted classification of an automated healthcare data analysis application to analyze healthcare data. In some non-limiting embodiments, AI data management system 102 may validate the machine learning model based on a validation threshold. For example, AI data management system 102 may be configured to validate the machine learning model when the classification of an automated healthcare data analysis application to analyze healthcare data (as identified by the validation data) are correctly predicted by the machine learning model (e.g., when the machine learning model correctly predicts 50% of the classifications of an automated healthcare data analysis application to analyze healthcare data, 70% of the classifications of an automated healthcare data analysis application, a threshold quantity of the classifications of an automated healthcare data analysis application, and/or the like).

In some non-limiting embodiments, if AI data management system 102 does not validate the machine learning model (e.g., when a percentage of correctly predicted classifications of automated healthcare data analysis applications does not satisfy the validation threshold), then AI data management system 102 may generate one or more additional machine learning models.

In some non-limiting embodiments, once the machine learning model has been validated, AI data management system 102 may further train the machine learning model and/or generate new machine learning models based on receiving new training data. The new training data may include additional healthcare data associated with classifications of automated healthcare data analysis applications. In some non-limiting embodiments, the new training data may include new healthcare data associated with a plurality of patient procedures (e.g., healthcare data associated with a plurality of patient procedures that were performed following a specified time). AI data management system 102 may use the machine learning model to predict classifications of automated healthcare data analysis applications and compare an output of machine learning models to the new training data that includes healthcare data associated with classifications of automated healthcare data analysis applications. In such an example, AI data management system 102 may update one or more machine learning models based on the new training data.

In some non-limiting embodiments, AI data management system 102 may store the machine learning model. For example, AI data management system 102 may store the machine learning model in a data structure (e.g., a database, a linked list, a tree, and/or the like). The data structure may be located within AI data management system 102 or external (e.g., remote from) AI data management system 102.

As shown in FIG. 3, at part 304, process 300 includes determining a classification of the healthcare data. For example, AI data management system 102 may determine the classification of the healthcare data using a machine learning model. The machine learning model may be configured to provide an output, where the output includes a predicted classification of an automated healthcare data analysis application of a plurality of automated healthcare data analysis applications, based on an input to the machine learning model. In some non-limiting embodiments, the input may include the healthcare data. Additionally or alternatively, AI data management system 102 may activate a set of clinical workflows based on the predicted classification of the automated healthcare data analysis application.

In some non-limiting embodiments, the machine learning model may be configured to receive a data record (e.g., a data record that includes healthcare data) associated with a patient as an input. In some non-limiting embodiments, the data record may include at least one feature. In some non-limiting embodiments, the at least one feature may include at least one feature associated with anatomical aspects of a body (e.g., a body of a patient), at least one feature associated with a protocol of a device (e.g., a device of a fluid injection system), at least one feature associated with a characteristic of natural language processing, and/or at least one feature associated with a manual configuration of a device (e.g., a medical device, a device associated with data source 104, etc.). In some non-limiting embodiments, AI data management system 102 may provide (e.g., generate, determine, etc.) one or more features. For example, AI data management system 102 may perform a feature extraction technique on healthcare data (e.g., healthcare data included in a data record) to provide the one or more features.

In some non-limiting embodiments, the machine learning model may be configured to determine whether a feature includes a feature of a category associated with anatomical aspects of a body, a feature of a category associated with a protocol of a device, a feature of a category associated with a characteristic of natural language processing, and/or a feature of a category associated with a manual configuration of a device. In some non-limiting embodiments, the machine learning model may determine a classification of the healthcare data (e.g., a classification associated with an automated healthcare data analysis application) based on determining whether the at least one feature includes a feature of a category associated with anatomical aspects of a body, a feature of a category associated with a protocol of a device, a feature of a category associated with a characteristic of natural language processing, or a feature of a category associated with a manual configuration of a device.

As shown in FIG. 3, at part 306, process 300 includes providing the healthcare data to an automated healthcare data analysis application based on the classification of the healthcare data. For example, AI data management system 102 may provide the healthcare data to an automated healthcare data analysis application (e.g., AI based healthcare data analysis application) based on the classification of the healthcare data. In some non-limiting embodiments, AI data management system 102 may provide the healthcare data to AI application system 108, and AI application system 108 may select an automated healthcare data analysis application of a plurality of automated healthcare data analysis applications based on the classification of the healthcare data. In some non-limiting embodiments, AI application system 108 may transmit the healthcare data to destination system 106 based on selecting the automated healthcare data analysis application of the plurality of automated healthcare data analysis applications. In some non-limiting embodiments, destination system 106 may be associated with the automated healthcare data analysis application that was selected by AI application system 108. In some non-limiting embodiments, AI application system 108 may transmit an output of the automated healthcare data analysis application to destination system 106.

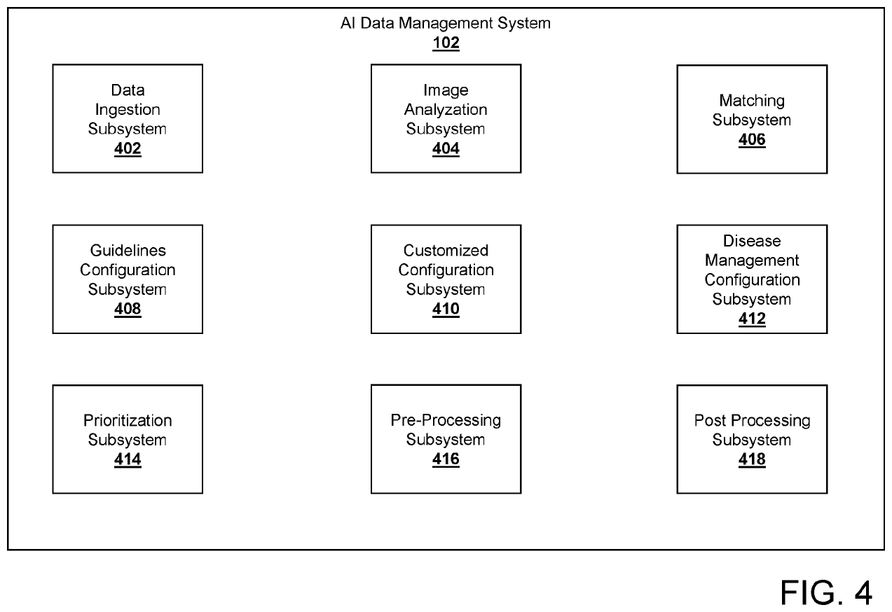

Referring now to FIG. 4, FIG. 4 is a diagram of a non-limiting embodiment of AI data management system 102. In some non-limiting embodiments, AI data management system 102 may include a plurality of subsystems (e.g., hardware components, software components, such as modules, applications, extensions, units, etc., that are configured to be executed on one or more devices of AI data management system 102 or any combination thereof). In some non-limiting embodiments, the plurality of subsystems of AI data management system 102 may interconnect with each other via wired connections, wireless connections, or a combination of wired and wireless connections.

As shown in FIG. 4, the plurality of subsystems may include data ingestion subsystem 402, image analyzation subsystem 404, matching subsystem 406, guidelines configuration subsystem 408, customized configuration subsystem 410, disease management configuration subsystem 412, and prioritization subsystem 414. In some non-limiting embodiments, one or more subsystems of the plurality of subsystems of AI data management system 102 may be hosted on a network device (e.g., a network edge device) that is closest to a facility that interacts with AI data management system 102. In this way, results from the one or more subsystems of AI data management system 102 may be received in less time as compared to a situation where the one or more subsystems are hosted on a different network device. In some non-limiting embodiments, AI data management system 102 may include more than or less than the plurality of subsystems shown in FIG. 4. In some non-limiting embodiments, AI data management system 102 may include one or more subsystems that are configured to be operated based on manually provided configuration (e.g., a configuration provided by a user, such as a doctor, technician, etc.).

In some non-limiting embodiments, data ingestion subsystem 402 may be configured to receive healthcare data from one or more data sources (e.g., one or more data sources 104) and data ingestion subsystem 402 may store, format, and/or perform additional functions on the healthcare data. In some non-limiting embodiments, data ingestion subsystem 402 may identify a patient associated with the healthcare data and/or match the healthcare data with a patient record (e.g., based on a patient name, a patient identification number, etc.).

In some non-limiting embodiments, image analyzation subsystem 404 may be configured to provide anatomical intelligence with regard to the healthcare data received by data ingestion subsystem 402. For example, image analyzation subsystem 404 may detect a body part (e.g., a body region) of a patient, analyze the image of the patient, and/or extract information from the image of the patient. In some non-limiting embodiments, image analyzation subsystem 404 may provide an output that includes an indication of one or more body parts and/or an indication of a characteristic (e.g., a quality) of an image. In some non-limiting embodiments, image analyzation subsystem 404 may determine a characteristic of an image (e.g., a type of image that identifies a body part of which the image is taken along with the modality of the image, such as a CT, an MRI, etc.). For example, image analyzation subsystem 404 may identify a body part portrayed in an image of a patient, determine whether a procedure involved the use of contrast (e.g., that the procedure involved the use of a contrast fluid or that the procedure did not involve contrast fluid), determine information about an image (e.g., slice thickness, pixel size, radiation dosage, voltage and/or amperes used, and other imaging parameters), extract text (e.g., DICOM text) that is included in an image, and determine an imaging protocol that was used to generate an image, to provide a characteristic of the image.

In some non-limiting embodiments, matching subsystem 406 may be configured to receive an output from image analyzation subsystem 404 and match the output to one or more (e.g., a set) of automated healthcare data analysis applications that are applicable for a characteristic of an image. For example, matching subsystem 406 may determine one or more automated healthcare data analysis applications of a plurality of automated healthcare data analysis applications that are to be used to analyze the output based on the characteristic of the image (e.g., an identification of a body part and a modality of the image). In some non-limiting embodiments, matching subsystem 406 may provide an output that includes a list of one or more automated healthcare data analysis applications (e.g., potential automated healthcare data analysis applications that may be run) and/or a plurality of input parameters for providing to the one or more automated healthcare data analysis applications (e.g., all automated healthcare data analysis applications included in a list of automated healthcare data analysis applications).

In some non-limiting embodiments, guidelines configuration subsystem 408 may be configured to compare the output of matching subsystem 406 and identify and/or select one or more automated healthcare data analysis applications of a plurality of automated healthcare data analysis applications (e.g., a plurality of automated healthcare data analysis applications identified by matching subsystem 406 that are to be used to analyze healthcare data, such as an image). In some non-limiting embodiments, guidelines configuration subsystem 408 may refine (e.g., remove, add, override, and/or the like, an indication of one or more automated healthcare data analysis applications) an output of matching subsystem 406 by comparing the output of matching subsystem 406 to one or more guidelines (e.g., default guidelines, such as industry standard guidelines that may be defined according to specified levels of patient care, regional guidelines that are specific to a particular geographic area, customer specified guidelines, etc.). In some non-limiting embodiments, the guidelines may be based on one or more automated healthcare data analysis applications that are associated with (e.g., available to and/or accessible to) a facility (e.g., a hospital). In some non-limiting embodiments, matching subsystem 406 may provide an output that includes a list of one or more automated healthcare data analysis applications (e.g., potential automated healthcare data analysis applications that may be run according to guidelines of guidelines configuration subsystem 408) and/or a plurality of input parameters for providing to the one or more automated healthcare data analysis applications (e.g., all automated healthcare data analysis applications included in a list of automated healthcare data analysis applications).

In some non-limiting embodiments, customized configuration subsystem 410 may be configured to compare the output of matching subsystem 406 and/or guidelines configuration subsystem 408 and identify and/or select one or more automated healthcare data analysis applications of a plurality of automated healthcare data analysis applications (e.g., a plurality of automated healthcare data analysis applications identified by matching subsystem 406 and/or guidelines configuration subsystem 408, that are to be used to analyze healthcare data, such as an image). In some non-limiting embodiments, customized configuration subsystem 410 may refine an output of matching subsystem 406 and/or guidelines configuration subsystem 408 by comparing the output of matching subsystem 406 and/or guidelines configuration subsystem 408 to one or more customized guidelines (e.g., individually specified guidelines). In some non-limiting embodiments, the customized guidelines may be based on one or more automated healthcare data analysis applications that are chosen to be associated with a facility based on predefined parameters (e.g., parameters that are specific to a facility, such as parameters associated with medical treatments that are available at a facility). In some non-limiting embodiments, matching subsystem 406 may provide an output that includes a list of one or more automated healthcare data analysis applications (e.g., potential automated healthcare data analysis applications that may be run according to guidelines of customized configuration subsystem 410) and/or a plurality of input parameters for providing to the one or more automated healthcare data analysis applications (e.g., all automated healthcare data analysis applications included in a list of automated healthcare data analysis applications).

In some non-limiting embodiments, disease management configuration subsystem 412 may be configured to provide a recommendation for a process (e.g., an additional automated healthcare data analysis application in addition to or as an alternative to outputs from guidelines configuration subsystem 408 and/or customized configuration subsystem 410) to analyze healthcare data based on one or more specified parameters (e.g., one or more specified parameters that are different from parameters used in guidelines of guidelines configuration subsystem 408 and/or customized configuration subsystem 410), such as a result that measures efficacy of an automated healthcare data analysis application, a disease tracking indication, and/or the like. In some non-limiting embodiments, disease management configuration subsystem 412 may provide an output that includes information associated with a measurement of an outcome (e.g., a patient outcome based on a patient procedure or treatment) and/or a recommendation, such as a recommendation for an opportunistic procedure (e.g., a medical screening).

In some non-limiting embodiments, prioritization subsystem 414 may be configured to determine a priority of automated healthcare data analysis applications (e.g., a plurality of automated healthcare data analysis applications that are determined to be applicable by matching subsystem 406, guidelines configuration subsystem 408, customized configuration subsystem 410, and/or disease management configuration subsystem 412) that are to be used to analyze healthcare data. In some non-limiting embodiments, prioritization subsystem 414 may receive a list of automated healthcare data analysis applications, and prioritization subsystem 414 may determine an order in which to run each of the automated healthcare data analysis applications in the list. In some non-limiting embodiments, prioritization subsystem 414 may determine the order in which to run each of the automated healthcare data analysis applications based on parameters, such as availability of an automated healthcare data analysis application (e.g., whether an automated healthcare data analysis application, an amount of time required for running an automated healthcare data analysis application, an automated healthcare data analysis application that was run prior (e.g., initially), a metric associated with severity of a patient, and/or the like)

In some non-limiting embodiments, pre-processing subsystem 416 may be configured to determine recommendation for a patient procedure (e.g., a scan) and/or a protocol (e.g., a protocol that includes scan parameters, such as slice thickness, a type of contrast fluid, an amount of contrast fluid, an amount of time for a procedure, a timing of different processes to be performed during a procedure, etc.) for the patient procedure before the patient procedure is performed. In some non-limiting embodiments, pre-processing subsystem 416 may determine the recommendation based on parameters, such as disease monitoring procedures for a patient, health conditions of a patient, prior treatment provided to a patient, information associated with one or more devices involved in a patient procedure, and/or the like. In some non-limiting embodiments, pre-processing subsystem 416 may provide a message (e.g., a report) that includes information regarding the recommendation to a device (e.g., a device associated with a hospital, such as a fluid injection device in a hospital, a device associated with medical personnel, such as a doctor and/or technician, a device associated with a treatment facility, etc.).

In some non-limiting embodiments, post processing subsystem 418 may be configured to receive an output of one or more automated healthcare data analysis applications and provide information (e.g., predictions, recommendations, conclusions, etc.) regarding one or more automated healthcare data analysis applications. For example, post processing subsystem 418 may provide information regarding one or more automated healthcare data analysis applications to another device (e.g., a user device associated with a user). In some non-limiting embodiments, post processing subsystem 418 may provide determinations about parameters (e.g., patient demographics, condition of a patient, disease treatment of a patient, etc.) that are used by one or more automated healthcare data analysis applications to provide an output. In some non-limiting embodiments, post processing subsystem 418 may provide information based on findings from a patient procedure, such as a scan, healthcare data, such as an image from a medical imaging procedure, results of a patient procedure, such a protocol variance (e.g., a protocol recommendation versus what was used in a protocol during a patient procedure), and/or the like. In some non-limiting embodiments, post processing subsystem 418 may provide a recommendation regarding guidelines (e.g., new guidelines, guidelines to remove, guidelines to emphasize, etc.) of guidelines configuration subsystem 408, customized configuration subsystem 410, and/or disease management configuration subsystem 412.

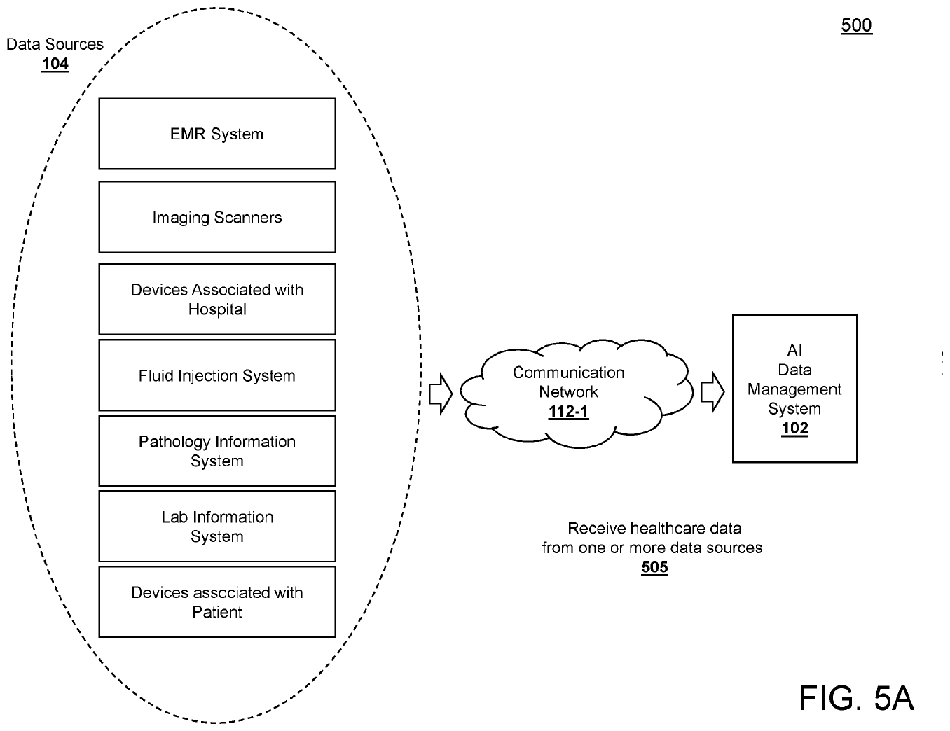

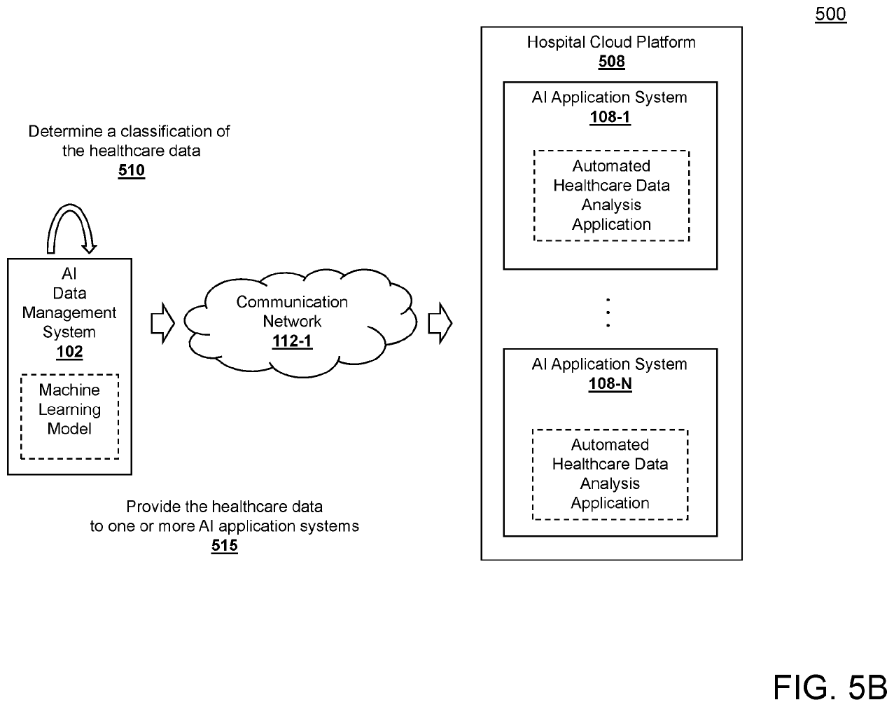

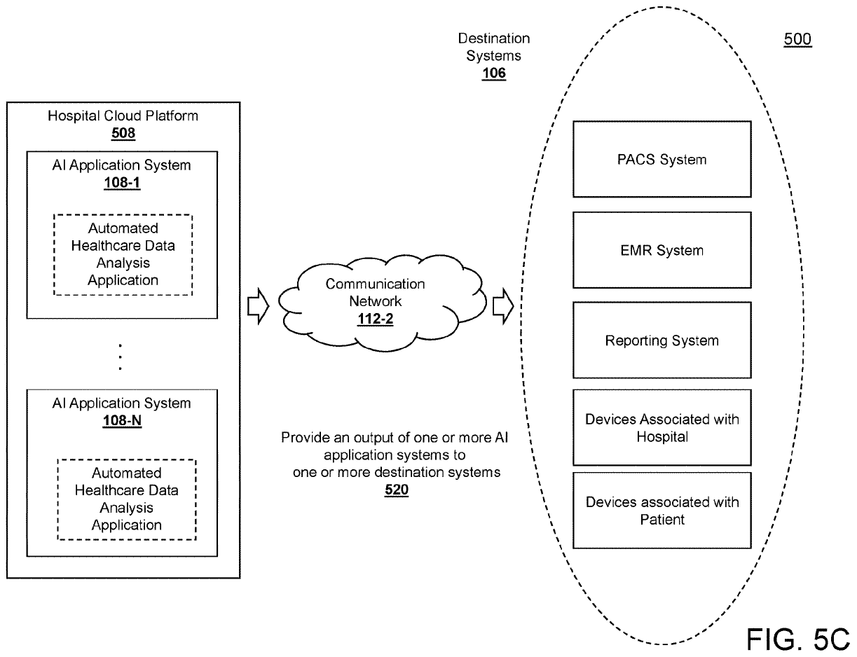

Referring now to FIGS. 5A-5C, FIGS. 5A-5C are diagrams of a non-limiting embodiment of implementation 500 of a process (e.g., process 300) for managing automated healthcare data analysis applications using AI. As shown in FIGS. 5A-5C, implementation 500 includes AI data management system 102, data sources 104, destination systems 106, hospital cloud platform 508 that includes AI application systems 108, communication network 112-1, and communication network 112-2. In some non-limiting embodiments, hospital cloud platform 508 may include an HIS that operated on a cloud platform. In some non-limiting embodiments, each AI application system 108 of AI application systems 108 may be associated with an automated healthcare data analysis application. For example, each AI application system 108 of AI application systems 108 may be configured to run an automated healthcare data analysis application based on healthcare data to provide an analysis (e.g., a prediction of a result of an analysis) of the healthcare data. In some non-limiting embodiments, communication network 112-1 and/or communication network 112-2 may be the same as or similar to communication network 112. In some non-limiting embodiments, communication network 112-1 and/or communication network 112-2 may be a communication network that is associated with (e.g., operated by) a hospital and/or may be located at (e.g., internal to) a hospital or other treatment facility. In some non-limiting embodiments, communication network 112-1 and communication network 112-2 may be the same (e.g., the same communication network).

As shown by reference number 505 in FIG. 5A, AI data management system 102 may receive healthcare data from one or more data sources. For example, AI data management system 102 may receive healthcare data from one or more devices or systems of data sources 104. In some non-limiting embodiments, AI data management system 102 may receive one or more data records from one or more data sources 104 that include the healthcare data.

As shown by reference number 510 in FIG. 5B, AI data management system 102 may determine a classification of the healthcare data. For example, AI data management system 102 may determine a classification of the healthcare data using a machine learning model (e.g., an automated healthcare data analysis application prediction machine learning model). In some non-limiting embodiments, AI data management system 102 may provide the healthcare data as an input to the machine learning model, and the machine learning model may generate an output based on the input. The machine learning model may provide a prediction of a classification of an automated healthcare data analysis application of a plurality of automated healthcare data analysis applications as an output.

As shown by reference number 515 in FIG. 5B, AI data management system 102 may provide the healthcare data to one or more AI application systems. For example, AI data management system 102 may provide the healthcare data to hospital cloud platform 508 or to one or more AI application systems 108 of hospital cloud platform 508. In some non-limiting embodiments, AI data management system 102 may provide the healthcare data to one or more AI application systems 108 based on the classification of the healthcare data. For example, AI data management system 102 may provide the healthcare data to AI application system 108 that is associated with an automated healthcare data analysis application that corresponds to the classification of the healthcare data. In this way, AI data management system 102 may provide the healthcare data to AI application system 108 that is appropriately configured to run an analysis of the healthcare data based on the automated healthcare data analysis application associated with AI application system 108. In some non-limiting embodiments, AI data management system 102 may provide the healthcare data to hospital cloud platform 508, and hospital cloud platform 508 may determine one or more AI application systems 108 of which to provide the healthcare data based on the classification of the healthcare data.

As shown by reference number 520 in FIG. 5C, hospital cloud platform 508 may provide an output of one or more AI application systems to one or more destination systems. For example, hospital cloud platform 508 may provide an output of one or more AI application systems 108 to one or more destination systems 106 based on the one or more AI application systems 108 running an automated healthcare data analysis application on the healthcare data. In some non-limiting embodiments, the output may include an output of an automated healthcare data analysis application. In some non-limiting embodiments, AI data management system 102 may cause hospital cloud platform 508 to provide the output of one or more AI application systems 108 to the one or more destination systems 106. For example, AI data management system 102 may control (e.g., via control signals transmitted to) hospital cloud platform 508 (e.g., one or more AI application systems 108 of hospital cloud platform 508) to cause hospital cloud platform 508 to provide the output of one or more AI application systems 108 to the one or more destination systems 106.

In some non-limiting embodiments, the one or more destination systems 106 may perform an action based on the output. For example, the one or more destination systems 106 may display the output, generate a report based on the output, transmit the output to another device (e.g., another destination system 106), provide an alert based on the output, generate a recommendation regarding a course of treatment based on the output, and/or the like.

Although the above systems, methods, and computer program products have been described in detail for the purpose of illustration based on what is currently considered to be the most practical and preferred embodiments, it is to be understood that such detail is solely for that purpose and that the present disclosure is not limited to the described embodiments but, on the contrary, is intended to cover modifications and equivalent arrangements that are within the spirit and scope of the appended claims. For example, it is to be understood that the present disclosure contemplates that, to the extent possible, at least one feature of any embodiment or aspect can be combined with at least one feature of any other embodiment or aspect.

What is claimed is:

1. A system for managing automated healthcare data analysis applications using artificial intelligence (AI), comprising:
at least one processor programmed or configured to:
receive healthcare data from a data source;
determine a classification of the healthcare data using a machine learning model, wherein the machine learning model is configured to provide a predicted classification of an automated healthcare data analysis application of a plurality of automated healthcare data analysis applications based on an input; and
provide the healthcare data to the automated healthcare data analysis application based on the classification of the healthcare data.

2. The system of claim 1, wherein the at least one processor is further programmed or configured to:
transmit an output of the automated healthcare data analysis application to a destination system.

3. The system of claim 2, wherein the destination system comprises:
a picture archiving and communication system (PACS);
an electronic medical record (EMR) system;
a data reporting system;
a communication device associated with a medical device; or
a user device associated with a patient.

4. The system of claim 1, wherein the at least one processor is further programmed or configured to:
train the machine learning model based on historic healthcare data from a plurality of data sources.

5. The system of claim 1, wherein the machine learning model is configured to receive a data record as an input, wherein the data record comprises at least one feature, and wherein the at least one feature comprises:
at least one feature associated with anatomical aspects of a body;
at least one feature associated with a protocol of a device;
at least one feature associated with a characteristic of natural language processing;
at least one feature associated with a manual configuration of a device; or
any combination thereof.

6. The system of claim 5, wherein the machine learning model is configured to determine whether the at least one feature comprises:
a feature of a category associated with anatomical aspects of a body;
a feature of a category associated with protocol of a device;
a feature of a category associated with a characteristic of natural language processing;
a feature of a category associated with a manual configuration of a device; or
any combination thereof.

7. The system of claim 1, wherein the data source comprises:
an electronic medical record (EMR) system;
a medical imaging system;
a communication device associated with a medical device;
a fluid injection system;
a pathology information system;
a laboratory information system; or
a user device associated with a patient.

8. The system of claim 1, wherein the automated healthcare data analysis application comprises an AI based healthcare data analysis application.

9. A computer program product for managing automated healthcare data analysis applications using artificial intelligence (AI), the computer program product comprising at least one non-transitory computer-readable medium including one or more instructions that, when executed by at least one processor, cause the at least one processor to:
receive healthcare data from a data source;
determine a classification of the healthcare data using a machine learning model, wherein the machine learning model is configured to provide a predicted classification of an automated healthcare data analysis application of a plurality of automated healthcare data analysis applications based on an input; and
provide the healthcare data to the automated healthcare data analysis application based on the classification of the healthcare data.

10. The computer program product of claim 9, wherein the one or more instructions further cause the at least one processor to:
transmit an output of the automated healthcare data analysis application to a destination system.

11. The computer program product of claim 10, wherein the destination system comprises:
a picture archiving and communication system (PACS);
an electronic medical record (EMR) system;
a data reporting system;
a communication device associated with a medical device; or
a user device associated with a patient.

12. The computer program product of claim 9, wherein the one or more instructions further cause the at least one processor to:
train the machine learning model based on historic healthcare data from a plurality of data sources.

13. The computer program product of claim 9, wherein the machine learning model is configured to receive a data record as an input, wherein the data record comprises at least one feature, and wherein the at least one feature comprises:
at least one feature associated with anatomical aspects of a body;
at least one feature associated with a protocol of a device;
at least one feature associated with a characteristic of natural language processing;
at least one feature associated with a manual configuration of a device; or
any combination thereof.

14. The computer program product of claim 13, wherein the machine learning model is configured to determine whether the at least one feature comprises:
a feature of a category associated with anatomical aspects of a body;
a feature of a category associated with protocol of a device;
a feature of a category associated with a characteristic of natural language processing;
a feature of a category associated with a manual configuration of a device; or
any combination thereof.

15. The computer program product of claim 9, wherein the data source comprises:
an electronic medical record (EMR) system;
a medical imaging system;
a communication device associated with a medical device;
a fluid injection system;
a pathology information system;
a laboratory information system; or
a user device associated with a patient.

16. The computer program product of claim 9, wherein the automated healthcare data analysis application comprises an artificial intelligence (AI) based healthcare data analysis application.

17. A method for managing automated healthcare data analysis applications using artificial intelligence (AI), comprising:
receiving healthcare data from a data source;
determining a classification of the healthcare data using a machine learning model, wherein the machine learning model is configured to provide a predicted classification of an automated healthcare data analysis application of a plurality of automated healthcare data analysis applications based on an input; and
providing the healthcare data to the automated healthcare data analysis application based on the classification of the healthcare data.

18. The method of claim 17, further comprising:
transmitting an output of the automated healthcare data analysis application to a destination system.

19. The method of claim 17, further comprising:
training the machine learning model based on historic healthcare data from a plurality of data sources.

20. The method of claim 17, wherein the machine learning model is configured to receive a data record as an input, wherein the data record comprises at least one feature, and wherein the at least one feature comprises:
at least one feature associated with anatomical aspects of a body;
at least one feature associated with a protocol of a device;
at least one feature associated with a characteristic of natural language processing;
at least one feature associated with a manual configuration of a device; or
any combination thereof.
